(19) United States
(12) Patent Application Publication
(10) Pub. No.: US 2024/0350186 A1
(43) Pub. Date: Oct. 24, 2024

(54) SURGICAL TOOL FOR TRACKING INTO FIBROUS TISSUE

(71) Applicant: Osama MAHMOUD, Giza (EG)

(72) Inventor: Osama MAHMOUD, Giza (EG)

(21) Appl. No.: 18/553,263

(22) PCT Filed: Mar. 30, 2021
(86) PCT No.: PCT/EG2021/050003

§ 371 Date: Sep. 29, 2023

(51) Int. Cl. A61B 18/08; A61B 18/00

(52) U.S. Cl. CPC A61B 18/082; A61B 2018/00595; A61B 2018/00601

(57) ABSTRACT

Disclosed is a surgical tool for tracking into fibrous tissue which may be used in penile prosthesis implantation surgery. The surgical tool has a hollow metal tube with a cutting edge along the perimeter of its tip. The tool is advanced into the fibrous tissue in an oscillating/rotating fashion, cutting a core of fibrous tissue with minimal resistance, thereby establishing the track.

### TECHNICAL FIELD

Surgery

### Background Art

Penile prosthesis implantation (PPI) is a surgery for cases of erectile dysfunction, whereby a certain cylindrical device (penile prosthesis/PP) is inserted into the penis to restore erection. PP is implanted into two natural cylinders called the corpora cavernosa. In some conditions, the corpora cavernosa are filled up and blocked by abnormal pathological and form tissue called fibrous tissue ([FIG. 1]). A track needs to be created through this tissue in order to implant a PP. Trying to establish the track using sharp instruments in a stabbing forward movement may result in perforations. Trying to establish the track using blunt instruments is difficult and may fail or also result in perforation. The resistance offered from the fibrous tissue may therefore prevent insertion of a PP, or make it very difficult or risky.

### SUMMARY OF INVENTION

We propose Shaeer's Cavernotome that helps to safely establish a track into the fibrous tissue. This is a hollow metal tube with a cutting edge at the tip. The cutting edge is in the form of triangular blades arranged around the permitter of the tip ([FIG. 2],2,3,5). Shaeer's cavernotome is advanced into the fibrous tissue in an oscillating/rotating fashion, cutting a core of fibrous tissue with minimal resistance ([FIG. 1]). Having established a track, the cavernotome is then pulled out, and PPI proceeds as usual.

### Technical Problem

Inability to make track to insert a penile prosthesis into the penis due to the presence of firm fibrous tissue

### Solution to Problem

Shaeer's Cavernotome can core out the fibrous tissue to make a track for implantation of a penile prosthesis into the penis

### Advantageous Effects of Invention

The risk involved with Shaeer's Cavernotome is much less than dilatation using blunt dilators, since the force needed with Shaeer's Cavernotome is minimal. The risk is also lower than using sharp scissors or scalpel, since with those, the tip is pushed against resistance in a stabbing forward movement that may result in perforation. In contrast, with Shaeer's cavernotome, the circular cutting tip is rotated gently as it cuts the core of fibrous tissue. The operative time is shorter, thereby saving the patient from possible infection and other complications of anesthesia.

### BRIEF DESCRIPTION OF DRAWINGS

FIG. 1 shows the problem that needs to be solved. FIGS. 2 through 5 show the design of Shaeer's Cavernotome. FIGS. 6 and 7 show variations of Shaeer's Cavernotome.

FIG. 1 Illustration of the penis with the erection cylinders (corpora cavernosa) blocked with fibrous tissue, and Shaeer's cavernotome introduced to core out the fibrous tissue

FIG. 2 Shaeer's cavernotome side view

FIG. 3 Shaeer's cavernotome top view

FIG. 4 Shaeer's cavernotome design—longitudinal section:
(1) Cutting Teeth: triangular metal blades arranged around the perimeter of the tip of the “tube”
(2) Tube: Hollow tube with the “cutting teeth at one end” and the “handle lock at the other”
(3) Handle Lock: A locking mechanism for attaching the “handle”
(4) Handle: The handle attaches to the “tube” to allow rotation of the tube. The “latch” locks into the “handle lock”. A spring secures the attachment of the “handle” to the “tube”
(5) Latch
(6) Spring

FIG. 5 Shaeer's cavernotome design-cross section

FIG. 6 Variation 1: Single Use Variant: The whole body (tube and handle) of Shaeer's Cavernotome can be manufactured from firm plastic or acrylic, with only the cutting teeth being metal. The cutting teeth can clip-on the tube in the manufacturing process as shown in the figure

FIG. 7 Variation 2: Variations in the teeth: The straight teeth will cut with half circle oscillating motion. This breaks fibrous tissue. The curved teeth will cut with full 360 rotation movement. This breaks fibrous tissue. The scalpel teeth cut a clean core of fibrous tissue and are easier to advance. Furthermore, the teeth can be directed slightly inwards

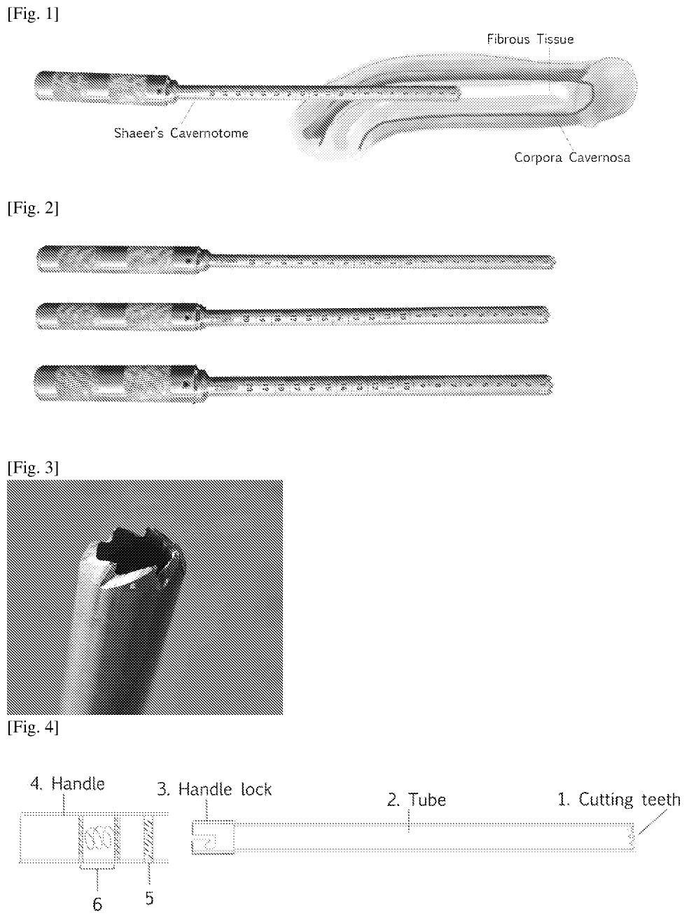

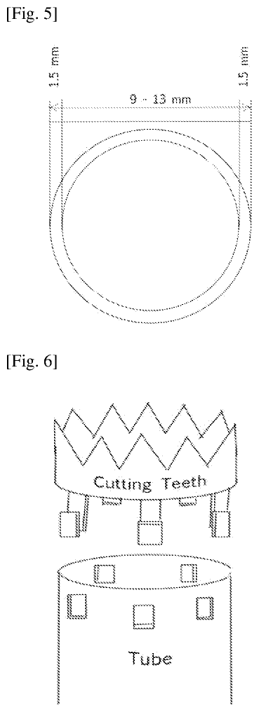

### DESCRIPTION OF EMBODIMENTS

Shaeer's cavernotome is a hollow tube with a cutting edge around the perimeter of one tip of the tube. It is used to cut/break-down/core our firm diseased tissue (fibrous tissue) during particular surgical procedures, namely penile prosthesis implantation ([FIG. 2],2,3,5). The metal tube is calibrated along its length in one-centimeter increments so that progress into the body can be measured. A handle can be attached to Shaeer's cavernotome other end to allow easy manipulation. The handle can be detached to clean the interior of the cavernotome. Shaeer's cavernotome is available in different girth measurements.

### Variations

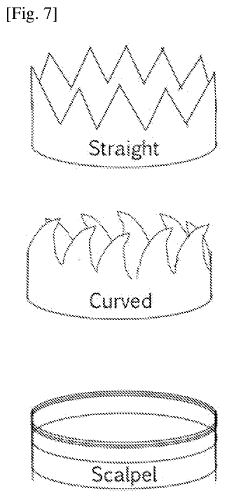

1. Single Use Variant: [FIG. 6]: The whole body (tube and handle) of Shaeer's Cavernotome can be manufactured from firm plastic or acrylic, with only the cutting teeth being metal. The cutting teeth can clip-on the tube in the manufacturing process
2. Variations in the teeth: [FIG. 7]: The straight teeth will cut with half circle oscillating motion. This breaks fibrous tissue. The curved teeth will cut with full 360 rotation movement. This breaks fibrous tissue. The scalpel teeth cut a clean core of fibrous tissue and are easier to advance. Furthermore, the teeth can be directed slightly inwards
3. Locking mechanism: The locking mechanism between the cavernotome and handle can either be with a clicking latch, or it can screw-on
4. Electro-cautery variant: Electro-cautery can be wired to the metal tip, with the insulated wire passing along the length of the tube. Electro-cautery applied to the tip will help cut through fibrous tissue with virtually no resistance.

### EXAMPLES

### INDUSTRIAL APPLICABILITY

In its simplest form, this is a surgical instrument in the form of a tube with a cutting circular tip. A handle attaches to it to enable manipulation. It can be manufactured from: metal as a reusable tool, or from medical grade plastics/acrylics or the like as a single-use instrument.

### REFERENCE SIGNS LIST

### REFERENCE TO DEPOSITED BIOLOGICAL MATERIAL

### SEQUENCE LISTING FREE TEXT

### CITATION LIST

### Patent Literature

PTL1:

### Non Patent Literature

NPL1:

1. A surgical instrument in the form of a hollow tube, having a sharp or cutting or serrated or jagged edge along a perimeter of a tip of the surgical instrument, to be used to break down or cut or advance into body tissues during surgical procedures.

2. The surgical instrument of claim 1, wherein the tube can accommodate insulated wiring to so that an electro-cautery current can be applied to the tip.
